(19) United States
(12) Patent Application Publication
(10) Pub. No.: US 2023/0318406 A1
(43) Pub. Date: Oct. 5, 2023

(54) WEARABLE DEVICE

(71) Applicant: SEIKO EPSON CORPORATION, Tokyo (JP)

(72) Inventors: Yoko YAMAZAKI, Ueda-shi (JP); Takeshi FUJISHIRO, Shiojiri-shi (JP)

(73) Assignee: SEIKO EPSON CORPORATION, Tokyo (JP)

(21) Appl. No.: 18/190,977

(22) Filed: Mar. 28, 2023

(30) Foreign Application Priority Data
Mar. 30, 2022 (JP) 2022-055306

(51) Int. Cl. H02K 11/20; H02J 50/10; H02K 5/16; H02K 11/30; A61B 5/024

(52) U.S. Cl. CPC H02K 11/20; A61B 5/02416; H02J 50/10; H02K 5/16; H02K 11/30; A61B 2560/0214; A61B 2562/0233

(57) ABSTRACT

A wearable device includes a first power generation module as a power generation module including a rotor with a first direction as an axial direction of the center of rotation, and a rotor bearing including a support portion configured to rotatably support the rotor, and a sensor configured to detect biological information and provided so as to overlap the support portion in the first direction.

The present application is based on, and claims priority from JP Application Serial Number 2022-055306, filed Mar. 30, 2022, the disclosure of which is hereby incorporated by reference herein in its entirety.

### BACKGROUND

### 1. Technical Field

The present disclosure relates to a wearable device that is attached to a body and detects biometric information by a sensor.

### 2. Related Art

For example, a power generation device-equipped watch has been known that is usable by being attached to a body as a wristwatch, and drives a hand of the watch by using the power generation device formed of a rotor and the like (JP-A-2004-264041) .

In contrast, in driving a wearable device that detects biometric information by a sensor, when an oscillating-type power generation device formed of the rotor and the like as installed in the power generation device-equipped watch exemplified in JP-A-2004-264041 described above is used as it is, detection of biometric information by the sensor may not be appropriately performed due to an influence of a load of rotating the rotor.

### SUMMARY

A wearable device according to one aspect of the present disclosure includes a power generation module including a rotor in which an axial direction of a rotation center is a first direction, and a rotor bearing including a support portion configured to rotatably support the rotor, and a sensor provided so as to overlap the support portion in the first direction and configured to detect biological information.

### BRIEF DESCRIPTION OF THE DRAWINGS

FIG. 1 is cross-sectional side view for describing an outline of a wearable device according to an embodiment.

FIG. 2 is a perspective view illustrating an appearance of the wearable device.

FIG. 3 is an exploded perspective view of the wearable device.

FIG. 4 is a diagram for describing a configuration of a first power generation module (power generation module) of the wearable device.

FIG. 5 is a conceptual diagram for describing a structure of a control board.

FIG. 6 is a table for describing an operation situation according to a mounting state of the wearable device.

FIG. 7 is a conceptual diagram for describing an aspect of charging (power generation) by a second power generation module.

FIG. 8 is cross-sectional side view for describing an outline of a wearable device according to one modification example.

### DESCRIPTION OF EXEMPLARY EMBODIMENTS

A wearable device of one embodiment according to the present disclosure will be described below with reference to the drawings.

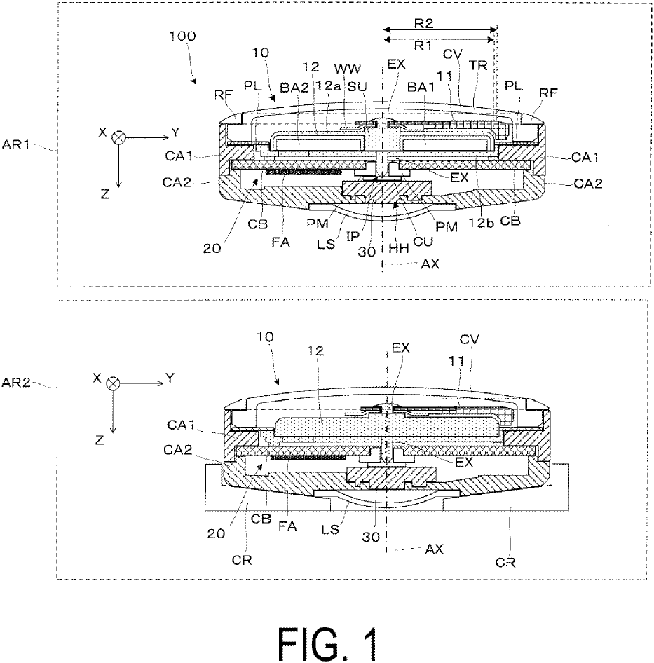

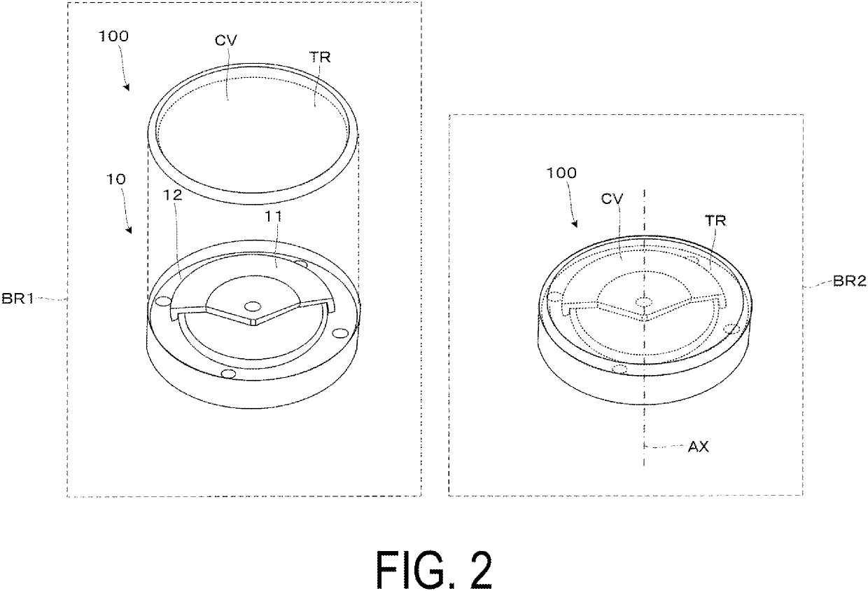

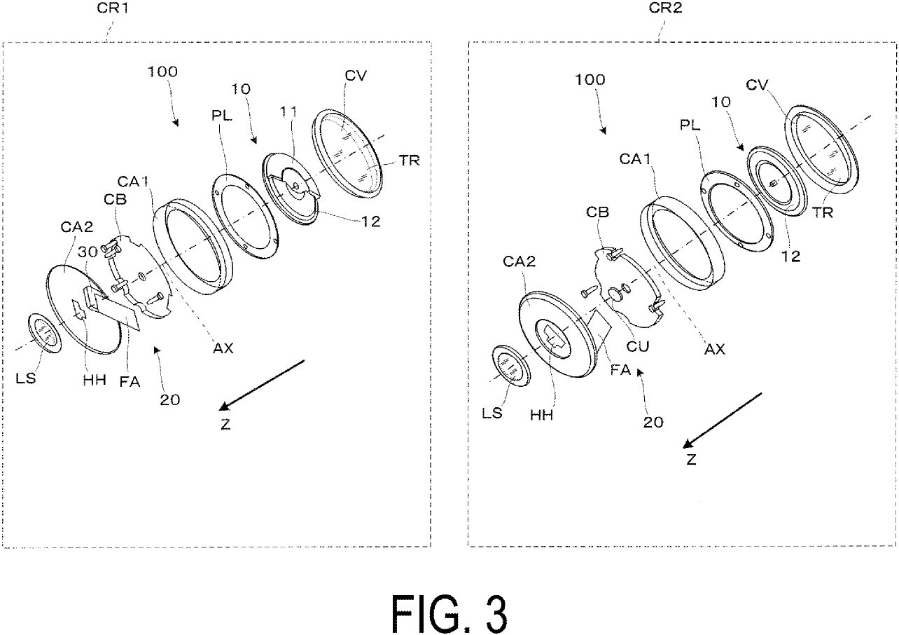

FIG. 1 is a conceptual diagram for describing a wearable device 100 according to the present embodiment. A state AR1 illustrates a conceptual cross-sectional side view of the wearable device 100, and a state AR2 illustrates the wearable device 100 installed on a cradle (placement table) CR. Further, FIG. 2 is a perspective view illustrating an appearance of the wearable device 100. A state BR1 of FIG. 2 illustrates a situation in which a cover member CV of the wearable device 100 that covers a front surface is removed, and a state BR2 of FIG. 2 illustrates a situation of the wearable device 100 to which the cover member CV is attached. Note that the cover member CV is formed of a light-transmitting member TR, and, as illustrated in the state BR2, the inside is visible through the light-transmitting member TR. Furthermore, FIG. 3 is an exploded perspective view of the wearable device 100. A state CR1 of FIG. 3 illustrates an exploded perspective view of the wearable device 100 viewed from one direction, and a state CR2 of FIG. 3 illustrates an exploded perspective view of the wearable device 100 viewed from another direction.

Note that, in FIG. 1 and the like, X, Y, and Z are an orthogonal coordinate system, and a +Z direction is a reference direction (thickness direction) in assembling the wearable device 100 and is a first direction. As illustrated (particularly, as illustrated in FIG. 3), units constituting the wearable device 100 are disposed side by side so as to overlap each other in the first direction. Further, an X direction and a Y direction are a direction perpendicular to the Z direction, most of the units constituting the wearable device 100 has a disc shape or a ring shape that isotropically extends along an XY plane, that is, a plane perpendicular to the Z direction, and has a thin (flat) cylindrical shape as the entire wearable device 100 as illustrated in FIG. 2. Note that, hereinafter, of the wearable device 100, a side being relatively the +Z side is a lower side of the wearable device 100, and a side being relatively a -Z side is an upper side of the wearable device 100.

The units constituting the wearable device 100 will be described below. First, as illustrated in FIG. 1 and the like, the wearable device 100 includes a first power generation module 10, a second power generation module 20, and a sensor 30. The first power generation module (power generation module) 10 is a power generation device that generates power by oscillation due to rotation of a rotor 11. Here, a central axis of the wearable device 100 having a cylindrical shape is an axis AX, and the rotor 11 rotates about the axis AX as the central axis. In other words, when a user (an operator, a wearer) of the wearable device 100 moves the wearable device 100 by wearing the wearable device 100 on his/her own arm, the rotor 11 rotates, thereby generating power. Note that the axis AX described above is an axis extending along the first direction (+Z direction). The second power generation module 20 is a power generation device that generates power by a magnetic force due to electromagnetic induction (more specifically, non-contact supply from the outside). The sensor 30 is a photoplethysmography (PPG) sensor, that is, an optical heart rate sensor for detecting biometric information. The sensor 30 is a pulse sensor module that performs detection for measuring a pulse being one piece of the biometric information by receiving return light that is irradiation light emitted toward the living body and reflected by a living body.

In order to achieve the configuration as described above, for example, as illustrated in FIG. 1, the wearable device 100 includes a rotor bearing 12, a control board CB, a supply antenna FA, a first case member CA1, a second case member CA2, a lens LS, and the like in addition to the rotor 11, the sensor 30, and the cover member CV. Note that, in one illustrated example, a power generator and the like for power generation, which are not illustrated, in addition to batteries BA1 and BA2 as secondary batteries are housed inside the rotor bearing 12 having a disc shape.

For example, as illustrated in, for example, FIG. 2 and the like, among these units, the rotor 11 has a fan shape or a semicircular shape with the axis AX as a position of a pivot, and is rotatably supported by a support portion SU formed of a portion of the rotor bearing 12 on a central side. As described above, the rotor 11 rotates about the first direction as an axial direction of the center of rotation. Note that, in one illustrated example, the rotor 11 is attached to an extending portion EX provided so as to extend in the ±Z direction (first direction) at the center of the support portion SU of the rotor bearing 12, and thus stable and high-efficiency axis rotation of the rotor 11 about the axis AX as the central axis can be achieved while suppressing an up-and-down movement in the Z direction. In other words, when a wearer (user) of the wearable device 100 performs a movement and the like, and the wearable device 100 oscillates, the rotor 11 rotates accordingly. Note that, as described above, the extending portion EX is provided so as to extend in the first direction at substantially the center of the rotor bearing 12, and is present in a region overlapping the support portion SU as viewed from the first direction.

Meanwhile, the rotor bearing 12 is provided on the lower side of the rotor 11 so as to rotatably support the rotor 11 as described above. Further, in one example herein, as described above, the rotor bearing 12 has a disc shape. Here, for the rotor bearing 12, a radius of the rotor bearing 12 indicated from a central position (position on the axis AX) having the disc shape to an edge portion is a first radius R1. On the other hand, for the rotor 11, a radius of the rotor 11 indicated from a position (position on the axis AX) of the center of rotation in the rotor 11 having the fan shape to an edge portion is a second radius R2. In this case, R2 > R1. In other words, the rotor 11 is a member having the second radius R2 greater than the first radius R1 of the rotor bearing 12. Further, the rotor bearing 12 is formed of an upper portion 12a constituting the upper side, that is, a side that supports the rotor 11, and a lower portion 12b constituting the lower side. As described above, the battery (secondary battery) BA1 and the like are provided between the upper portion 12a and the lower portion 12b. Rotation of the rotor 11 is transmitted, via a rotor wheel WW and the like, to the power generator (not illustrated) housed inside the rotor bearing 12 to generate power, and the generated power is stored in the battery BA1. In the case describe above, the first power generation module 10 that generates power by oscillation is formed of the rotor 11, the rotor bearing 12, and the like as a power generation module constituting the wearable device 100 in the present embodiment. Note that, in one illustrated example, the battery BA2 is separately provided, and power in the second power generation module 20 described below is stored in the battery BA2. Note that, when a perspective is changed for the aspect described above, the rotor bearing 12 is provided between the rotor 11 and the second power generation module 20 in the cross-sectional side view as illustrated in FIG. 1.

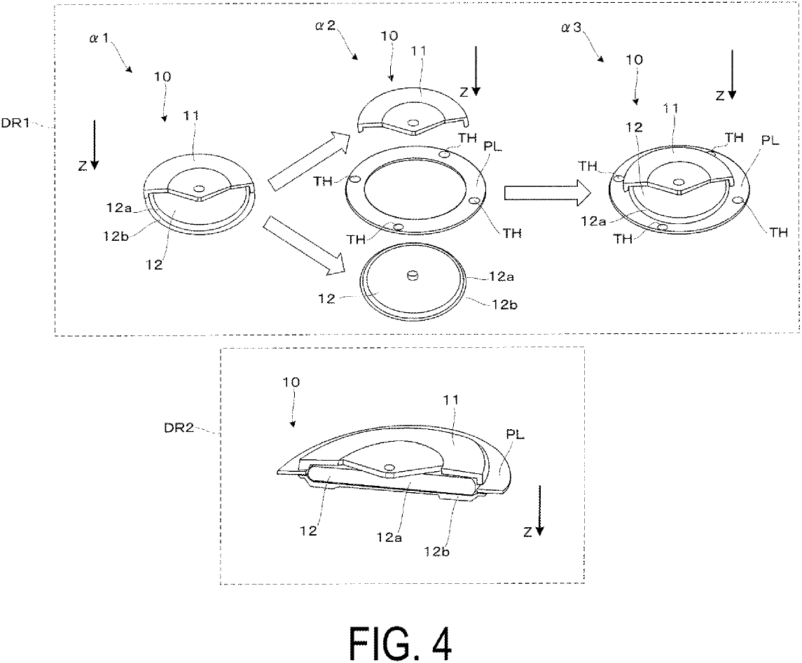

Further, a plate member PL having an annular shape formed of a metal plate is attached and fixed between the rotor 11 and the rotor bearing 12, and is provided for forming a peripheral edge portion of the rotor bearing 12. Note that one example will be described below with reference to FIG. 4 with regard to attachment of the plate member PL.

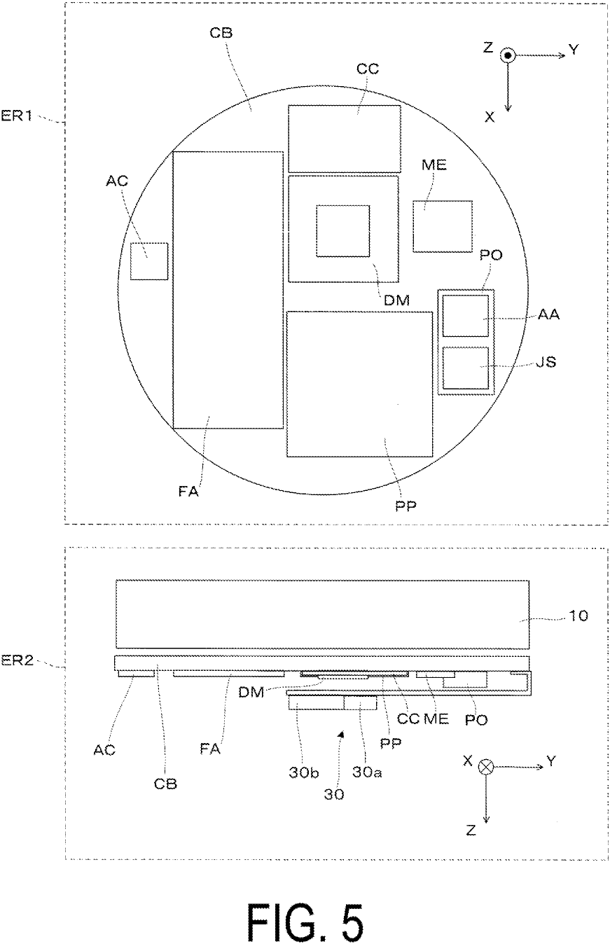

The control board CB is a member having a disc shape. Note that, in one illustrated example, an insertion port IP into which the extending portion EX of the rotor bearing 12 is inserted is provided in a central portion of the disc shape. Furthermore, an attachment member PM having a cylindrical shape formed of a resin accompanies the insertion port IP, and the extending portion EX is inserted into the insertion port IP so as to penetrate the attachment member PM. Note that, in this way, for example, the rotor bearing 12 and the like may be positioned with respect to the control board CB. The control board CB is formed of a CPU and the like in a main body portion having the disc shape described above, and performs various types of operation processing in the wearable device 100 such as supply, power supply to each unit, and recording of biometric information in addition to control of the sensor 30. Here, particularly, power supply to the sensor 30 is performed as the control of the sensor 30. In one illustrated example, the insertion port IP of the control board CB is disposed in a position on a central side overlapping the sensor 30 in the first direction. Further, although not illustrated, in order to be supplied with power, for example, on a side surface side on the disc shape, the control board CB has a contact with the rotor bearing 12, more accurately, the batteries BA1 and BA2 housed in the rotor bearing 12, that is, the control board CB is connected to the batteries BA1 and BA2 in a wired manner. As a result, the sensor 30 is supplied with power from the first power generation module 10 and the second power generation module 20 via the control board CB. Note that details of one configuration example of the control board CB will be described below with reference to FIG. 5.

The supply antenna FA is a near field communication (NFC) antenna formed of, for example, a loop coil and the like, and can receive a radio wave from the outside, but the supply antenna FA is connected to the control board CB, and performs non-contact supply using transmission by a transmission antenna from the outside according to control of the control board CB herein. In this way, although details are not illustrated, for example, an aspect in which power is stored in the battery BA2 can be achieved. In other words, the wearable device 100 can perform power generation by electromagnetic induction (by a magnetic force) by using the supply antenna FA. Further, in the case described above, the second power generation module 20 that generates power by the magnetic force is formed of the supply antenna FA, a processing unit of the control board CB that performs operation processing of non-contact supply, and the like.

Note that, although the transmission antenna from the outside of the units described above is not illustrated, for example, an aspect in which the transmission antenna is provided in the cradle CR exemplified as in the state AR2 in FIG. 1, and supply (charging) is performed by placing the wearable device 100 on the cradle CR during non-mounting time of the wearable device 100 can be achieved.

Note that, in addition to the description above, for example, the cover member CV is a member for covering the front surface, that is, an uppermost side of the wearable device 100. in one illustrated example, the cover member CV is formed of the light-transmitting member TR formed of glass or a resin, and a frame body RF having a ring shape provided on a peripheral side of the light-transmitting member TR.

Further, the first case member CA1 is, for example, a member of a frame body having a cylindrical shape formed of a resin, and is attached to a lower portion (+Z side) of the cover member CV (optical transparency member TR). The rotor 11 and the rotor bearing 12 constituting the first power generation module 10 are covered with the cover member CV and the first case member CA1. More specifically, the first power generation module 10 is attached to the first case member CA1 on the lower side (+Z side) while being covered with the cover member CV from the upper side (-Z side). Note that, in the case of the arrangement as described above, for example, the extending portion EX of the rotor bearing 12 of the first power generation module 10 is provided at substantially the center of the first case member CA1 as viewed from the first direction.

Further, the second case member CA2 is, for example, a member that is formed of a resin, has a shape in which an edge portion is provided on a disc, and further has a hole HH on a central side. The second case member CA2 is attached to the lower portion (+Z side) of the first case member CA1. The sensor 30, and the supply antenna FA and the control board CB constituting the second power generation module 20 are covered with the first case member CA1 and the second case member CA2. More specifically, for the second power generation module 20 and the sensor 30, while the second power generation module 20 is attached to the first case member CA1 on the upper side (-Z side), the sensor 30 is attached to the second case member CA2 on the lower side (+Z side).

Further, in the case described above, the first power generation module 10 is attached from one side (the -Z side, the upper side) of the first case member CA1, and the second power generation module 20 is attached from another side (the +Z side, the lower side).

As described above, in order to perform detection for measuring a pulse, the sensor 30 emits irradiation light toward a living body, and receives return light that is the irradiation light reflected by the living body. In order to accurately perform such an operation, the sensor 30 is installed so as to be able to emit the irradiation light in the first direction while being disposed on a central position or the axis AX. Specifically, in the configuration described above, the sensor 30 is installed so as to fit in the hole HH with respect to the first direction (Z direction), and the lens LS is provided on the second case member CA2 so as to protrude to the outside (lower side, +Z side) in a place corresponding to the hole HH. In this case, the sensor 30 and the lens LS are disposed so as to be aligned on the axis AX and overlap each other with respect to the first direction. As described above, the irradiation light emitted from the sensor 30 in the +Z direction is applied from a central position of a back surface of the wearable device 100 toward the outside, that is, toward a living body located on the lower side (+Z side) via the lens LS. Further, return light that is the irradiation light emitted toward the living body and reflected by the living body reaches the sensor 30 via the lens LS, and the sensor 30 receives the light. Note that, as a result of the arrangement as described above, the sensor 30 is provided so as to overlap the support portion SU of the rotor bearing 12 in the first direction.

Further, in one example in the cross-sectional side view in FIG. 1, a cushioning member CU is provided between the control board CB and the sensor 30. In the illustrated case, the insertion port IP of the control board CB or the attachment member PM accompanying the insertion port IP is present on the upper side of the sensor 30, and the cushioning member CU is attached so as to be sandwiched between the insertion port IP and the attachment member PM, and the sensor 30.

One specific aspect with regard to the configuration of the first power generation module 10 of the wearable device 100 will be further described below with reference to FIG. 4. Here, particularly, the plate member (metal sheet) PL in the first power generation module 10, and attachment of the plate member PL will be described. A state DR1 of FIG. 4 is a perspective view exemplifying a state of the attachment of the plate member PL with respect to the rotor 11 and the rotor bearing 12 constituting the first power generation module 10, and a state DR2 of FIG. 4 is a perspective cross-sectional view illustrating a state of the first power generation module 10 after the attachment of the plate member PL.

As exemplified as step α1 of the state DR1, the rotor 11 and the rotor bearing 12 in general may have a shape in which an attachment portion to another member cannot be sufficiently secured in a peripheral portion (edge portion) of the rotor 11 and the rotor bearing 12. More specifically, the aspect according to the present embodiment needs the attachment portion for attaching the first power generation module 10 from the one side (-Z side) of the first case member CA1. Thus, as illustrated, the present embodiment has a configuration in which the plate member PL having the annular shape and having a through hole TH for being screwed and fixed to another member is provided between the rotor 11 and the rotor bearing 12 with respect to the first direction. Specifically, as exemplified as step α2, the rotor 11 and the rotor bearing 12 without including the plate member PL as illustrated in step α1 are disassembled once, and, as exemplified as step α3, the rotor 11 and the rotor bearing 12 that have been disassembled are assembled again so as to sandwich the plate member PL therebetween to form the first power generation module 10 including the plate member PL as described above. Note that, in this case, the plate member PL forms the peripheral edge portion in the first power generation module 10, and, as illustrated in FIG. 1 and the like, the plate member PL is a member fixed to the first case member CA1.

One example of the configuration of the control board CB will be described below with reference to a conceptual diagram illustrated as FIG. 5. A state ER1 of FIG. 5 is a conceptual plan view of the control board CB, and a state ER2 of FIG. 5 is a conceptual side view of the control board CB and a peripheral portion of the control board CB.

As illustrated, in one example herein, the control board CB includes a data management unit DM, a memory (flash) ME, a communication antenna (Bluetooth low energy (BLE)) CC, a posture detection device PO, a power source circuit PP, and a supply antenna circuit AC in addition to the accompanying supply antenna FA. Furthermore, as illustrated in the state ER2, the sensor 30 is also connected to the control board CB, and the sensor 30 includes, for example, a driving circuit and the like, and is supplied with power from the control board CB side, and also performs a detection operation according to a command from the control board CB. Further, in one illustrated example, the sensor 30 includes a light-emitting unit 30a and a light-receiving unit 30b. In other words, the light-emitting unit 30a of the sensor 30 emits irradiation light toward a living body. Meanwhile, the light-receiving unit 30b receives return light that is the irradiation light emitted from the light-emitting unit 30a and reflected by the living body. Note that at least one of the light-emitting unit 30a or the light-receiving unit 30b is disposed in a position at substantially the center overlapping the support portion SU (see FIG. 1) of the rotor bearing 12 in the first direction. As described above, the wearable device 100 acquires and manages data about the emission from the light-emitting unit 30a and data about the reception of the return light in the light-receiving unit 30b while controlling an operation of the light-emitting unit 30a and the light-receiving unit 30b constituting the sensor 30.

In order to perform the operation as described above, for example, the data management unit DM of the wearable device 100 is formed of, for example, a memory control unit (MCU) and the like, and manages various types of data about biometric information acquired by sensing by the sensor 30.

The memory ME is formed of, for example, a storage device such as a flash, and stores data to be a target acquired and managed according to an instruction of the data management unit DM.

The communication antenna CC is, for example, an antenna for performing near field communication by extremely low power such as BLE, and transmits various types of data about biometric information accumulated in the memory ME to the outside.

The posture detection device PO is a device for detecting a posture (motion) of the wearable device 100, and is formed of an acceleration sensor AA and a gyro sensor JS in one illustrated example. In a state where the wearable device 100 is mounted on a user (an operator, a wearer), when the user starts a movement, the wearable device 100 also starts moving accordingly. In the posture detection device PO, whether the user is currently moving or resting can be determined by capturing such a motion, that is, a change in the posture. Further, as a usage aspect of the wearable device 100, it is determined that a movement starts when a specific motion is detected from the acceleration sensor AA and the gyro sensor JS, and biometric information acquisition by the sensor 30 during the movement can start with the determination as a trigger.

The power source circuit PP is a circuit for stably supplying power needed in an operation of each unit as described above, and is formed of a capacitor and the like. The power source circuit PP can stably continue the operation of the biometric information acquisition by using power stored in not only the battery BA1 but also the battery BA2 (see FIG. 1).

Note that the supply antenna circuit AC is a circuit for controlling an operation of the supply antenna FA. As described above, during non-mounting time when a user does not use the wearable device 100, supply (charging) from the outside is performed on the supply antenna FA in a state where the wearable device 100 is placed on the cradle CR. At this time, the supply antenna circuit AC controls an operation of power supply by the supply antenna FA, and controls an operation for storage in the battery BA2 (see FIG. 1).

With the aspect as described above, during the non-mounting time, power generation (supply) is performed on the second power generation module 20 formed of the supply antenna FA and the like in the wearable device 100, thereby accumulating power. On the other hand, during mounting time, the wearable device 100 moves together with a user wearing the wearable device 100, and thus oscillation occurs, and power generation is accordingly performed in the first power generation module 10, thereby accumulating power. With an aspect in which the wearable device 100 is driven based on both of power generation in the first power generation module 10 and power generation in the second power generation module 20, occurrence of a power shortage can be further suppressed, and stable detection on a living body can continue.

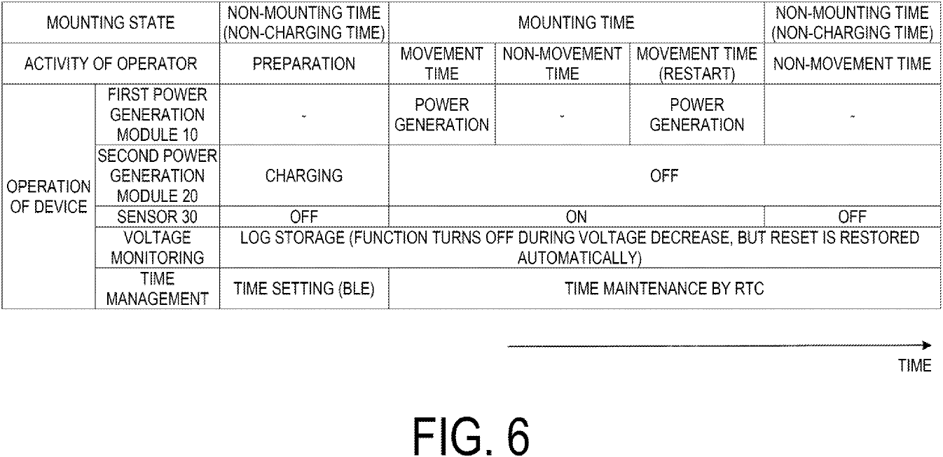

One example of an operation situation according to a mounting state of the wearable device 100 will be described below with reference to FIG. 6.

A table illustrated in FIG. 6 illustrates a change in the operation situation in time series before, during, and after mounting of the wearable device 100 by a user of the wearable device 100, and a horizontal direction in the table is along a time flow. Here, as one example of a specific usage aspect, first, at a point in time before a movement starts (before the wearable device 100 is mounted), the wearable device 100 is placed on the cradle CR, and charging (power generation, supply) in the second power generation module 20 is performed. Subsequently, a user wears the wearable device 100 and starts a movement, and a measurement (detection of biometric information) by the wearable device 100 also stars. For example, after a movement for about an hour is performed, a rest is taken for a while (non-movement time), and, after the rest, a movement restarts, and, for example, a movement for about an hour is performed and the movement ends, and the measurement by the wearable device 100 also ends. Such an operation aspect is assumed as one example. Note that a measurement (detection) result is recorded in the memory ME (see FIG. 5) in the wearable device 100 until a movement ends, and various types of the recorded data are collectively transmitted to the outside via the communication antenna CC after the movement ends.

Details of the aspect described above will be described below in time series. First, before mounting, that is, during non-mounting time, for example, the wearable device 100 is placed on the cradle CR, and thus charging is performed. In other words, power is accumulated by power generation (supply) in the second power generation module 20. On the other hand, in this case, oscillation does not occur, and power generation in the first power generation module 10 is not performed. Further, in this case, the sensor 30 is not also operated, and an acquisition operation (measurement of a pulse) of biometric information is not performed.

Subsequently, when the wearable device 100 is removed from the cradle CR, and a user wears the wearable device 100 and starts up the wearable device 100 (mounting time) by appropriately performing an operation, a movement of the user starts (movement time), and power is also accumulated by the power generation in the first power generation module 10 in response to oscillation of the wearable device 100. On the other hand, in this case, the power generation (supply) in the second power generation module 20 is not performed. Even during the mounting time, when a user stops a movement and takes a rest (non-movement time), oscillation does not occur, and the power generation in the first power generation module 10 is not performed. When a movement restarts, the power generation in the first power generation module 10 starts again. However, the sensor 30 continues to perform sensing even during the movement time and the non-movement time. In other words, during the mounting time, the acquisition operation (measurement of a pulse) of biometric information continues to be performed regardless of during moving or resting. Note that, as described above, for example, an aspect in which whether the movement time or the non-movement time is determined by using the posture detection device PO (see FIG. 5) is conceivable. Alternatively, for example, a button or the like (not illustrated) may be provided on a side surface portion of the wearable device 100 having a disc shape, and may be pressed by a user to switch between the movement time and the non-movement time.

Subsequently, when the user finishes the movement, and performs an operation for stopping an operation of the wearable device 100 in order to finish the measurement of the pulse, an operation of the sensor 30 is stopped. Note that, in this case, for example, before the wearable device 100 is placed on the cradle CR, both of the power generation in the first power generation module 10 and the power generation (supply) in the second power generation module 20 are not performed.

However, in order to confirm an operation situation of the wearable device 100, voltage monitoring in the wearable device 100 continues from beginning to end throughout the operation above. In other words, log storage as the voltage monitoring since charging before mounting until an end of a movement continues to be performed except for an interval between a decrease in voltage and restoration.

In addition, for time management performed together with an operation for acquisition of various types of the data described above, for example, during charging before mounting, absolute time setting based on management in an external device (not illustrated) is performed by using near field communication by the communication antenna CC. However, during the mounting time, time maintenance by a real-time clock (RTC) provided inside the wearable device 100 is used.

Note that the operation aspect described above is one example, and can be changed to various aspects. For example, for power generation in the first power generation module 10, an aspect in which power generation due to oscillation continues to be performed even before the wearable device 100 starts up and after an operation of the wearable device 100 is stopped is also conceivable.

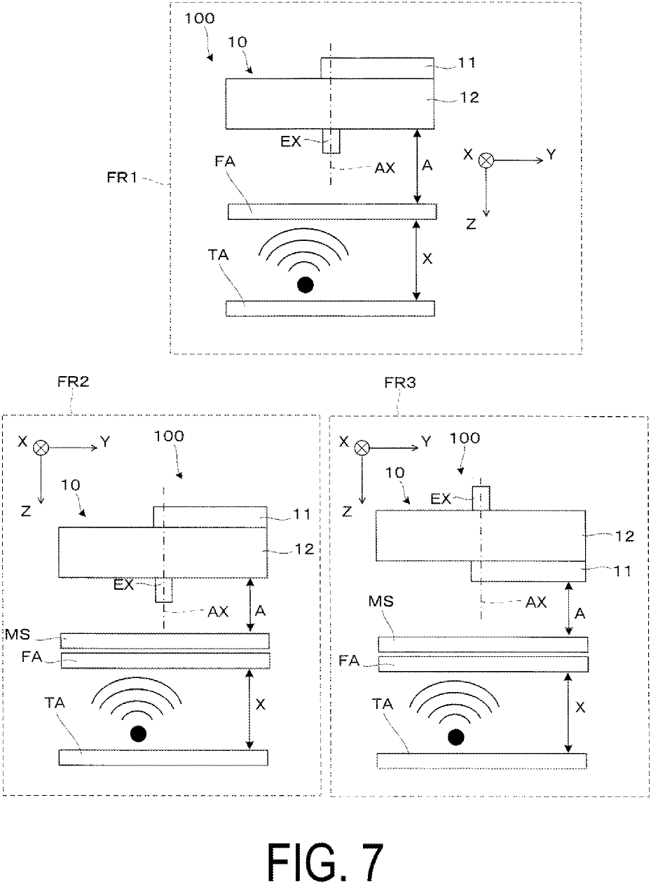

An aspect of charging (power generation, supply) by the second power generation module 20 will be described below with reference to a conceptual cross-sectional side view illustrated as FIG. 7. The charging by the second power generation module 20 uses electromagnetic induction, that is, a magnetic force (change in a magnetic field). In this case, for example, a possibility and the like that rotation and the like of the rotor 11 in the first power generation module 10 affect a magnetic field are conceivable.

Thus, here, in the charging (power generation, supply) by the second power generation module 20, particularly, a distance from the first power generation module 10, and provision of a ferromagnetic sheet between the first power generation module 10 and the second power generation module 20 will be considered, and a positional relationship between the rotor 11 and the rotor bearing 12 constituting the first power generation module 10 will be further considered.

First, in one example illustrated in a state FR1 of FIG. 7, in cross-sectional side view, the supply antenna FA constituting the second power generation module 20 is disposed at an appropriate distance X (for example, X = 10 mm) from a transmission antenna TA being a supply source of power with respect to the first direction (Z direction). Furthermore, a distance A from the first power generation module 10 to the second power generation module 20 is set to be equal to or more than 2 mm. Note that an antenna suitable for supply associated with the configuration of the supply antenna FA can be appropriately adopted as the transmission antenna TA. Further, in one example according to the present embodiment described above, as illustrated, the rotor 11 of the first power generation module 10 that can rotate is disposed on a side (the -Z side) farther from the second power generation module 20 than the fixed rotor bearing 12.

With the arrangement relationship as described above, it is clear that the charging (power generation, supply) by the second power generation module 20 can be stably performed.

Further, as another aspect, in one example illustrated in a state FR2 of FIG. 7, the wearable device 100 is configured to include a ferromagnetic sheet MS between the first power generation module 10 and the second power generation module 20 (supply antenna FA). Here, a ferrite sheet is used as one example of the ferromagnetic sheet MS. Note that, also in this aspect, similarly to the case of one example illustrated in the state FR1, the rotor 11 is disposed on the side (-Z side) farther from the second power generation module 20 than the rotor bearing 12.

Also, with the configuration as described above, it is clear that the charging (power generation, supply) by the second power generation module 20 can be more stably performed. Particularly, in this case, it is clear that the distance A can be set to be equal to or less than 2 mm. Note that, in the illustration, the ferromagnetic sheet (ferrite sheet) MS is illustrated so as to be separately independent of the supply antenna FA, but a configuration in which the ferrite sheet as the ferromagnetic sheet MS is bonded to the supply antenna FA is conceivable.

As still another aspect, in one example illustrated in a state FR3 of FIG. 7, the state of one example illustrated in the state FR2, that is, the state where the ferromagnetic sheet MS is provided is changed to a state where the positional relationship between the rotor 11 and the rotor bearing 12 is reversed. In other words, an arrangement is changed such that the rotor 11 is located on a side (the +Z side) closer to the second power generation module 20 than the rotor bearing 12. Also, with the configuration as described above, it is clear that that charging (power generation, supply) by the second power generation module 20 can be stably performed by setting the distance A to be equal to or more than 2 mm.

However, it is also clear that, in a case in which the state of one example illustrated in the state FR3 is changed to a configuration without using the ferromagnetic sheet MS, the charging (power generation, supply) by the second power generation module 20 cannot be normally performed even when the distance A is set to 2 mm.

As described above, in the configuration according to the present embodiment, the distance A is set to be equal to or more than 2 mm, or the ferromagnetic sheet MS is inserted between the second power generation module 20 (supply antenna FA) and the first power generation module 10. In this way, in the configuration including the first power generation module 10, the charging (power generation, supply) by the second power generation module 20 can be stably maintained.

Note that, for example, from a viewpoint of performing a movement with the wearable device 100 mounted on an arm, in terms of maintaining a reduction in thickness (size) for mountability, it is conceivably desirable that an upper limit of the distance A is set within about, for example, 10 mm. Further, the distance X is appropriately set according to the adopted supply antenna FA and the adopted transmission antenna TA.

As described above, the wearable device 100 according to the present embodiment includes the first power generation module 10 as a power generation module including the rotor 11 with the first direction as the axial direction of the center of rotation, and the rotor bearing 12 including the support portion SU configured to rotatably support the rotor 11, and the sensor 30 configured to detect biological information and provided so as to overlap the support portion SU in the first direction. In the wearable device 100 described above, in a structure in which the sensor 30 can be supplied with power from the first power generation module 10 that generates power by rotation of the rotor 11, the sensor 30 is provided so as to overlap the support portion SU of the rotor bearing 12 that rotatably supports the rotor 11 in the first direction being the axial direction of the center of rotation. In this way, occurrence of a position shift and the like of the sensor 30 due to a load of rotating the rotor 11 can be suppressed, and detection of appropriate biometric information can be maintained.

Note that, in the present embodiment, an environment is considered by the configuration in which supply by the second power generation module 20 from the outside and a power generation technology using oscillation of the wearable device 100 are combined.

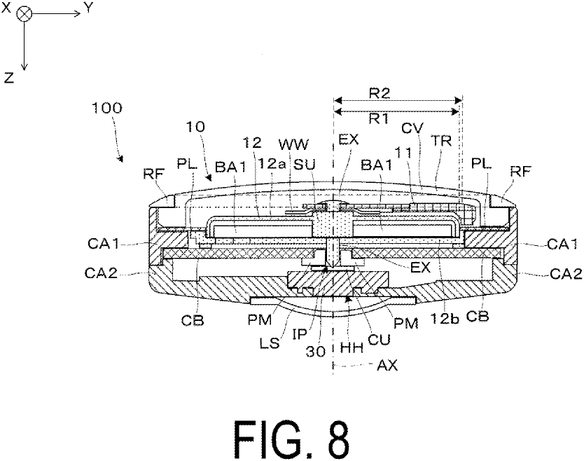

An outline of the wearable device 100 according to one modification example will be described below with reference to FIG. 8. Note that FIG. 8 corresponds to the diagram illustrated as the state AR1 of FIG. 1.

The present modification example is different from the aspect of one example described above in a configuration without including the second power generation module 20. In other words, as clear when the wearable device 100 according to the present modification example exemplified in FIG. 8 is compared with the case illustrated in FIG. 1, the wearable device 100 has a configuration in which the supply antenna FA constituting the second power generation module 20, and the battery BA2 that accumulates power generated in the second power generation module 20 are not provided. Note that the configuration other than this point is similar to that in the case described with reference to FIG. 1 and the like, and thus description will be omitted.

Also, in the case of the aspect described above, in the first power generation module 10, an operation of sensing by the sensor 30 can be achieved by using power generated due to rotation of the rotor 11. Further, also in this case, a position shift and the like of the sensor 30 due to a load of rotating the rotor 11 can be suppressed by providing the sensor 30 so as to overlap the support portion SU that rotatably supports the rotor 11 in the first direction being the axial direction of the center of rotation.

Note that, in the present modification example, in addition to a case in which a power supply source is only the first power generation module 10, a configuration in which a power supply source of another aspect different from the second power generation module 20 is separately provided is also conceivable. For example, a configuration in which a contact-type charging facility is provided is conceivable. In the case of the aspect in which the power supply source is only the first power generation module 10, an environment is further considered.

### Modified Examples and Others

The present disclosure is described according to the above-described embodiments, but the present disclosure is not limited to the above-described embodiments. The present disclosure may be carried out in various modes without departing from the gist of the present disclosure, and, for example, the following modifications may be carried out.

In the wearable device 100 according to each of the embodiments described above, the first power generation module 10 has the configuration in which the extending portion EX is provided at the center of the support portion SU of the rotor bearing 12. However, the present disclosure is not limited to this, and various aspects can be achieved as long as rotation of the rotor 11 due to oscillation is appropriately maintained, and a configuration in which the extending portion EX is not included is also conceivable. Further, when the extending portion EX is not included, the insertion port IP may not also be provided in the control board CB.

Further, in the description described above, the battery BA1 that performs storage due to power generation in the first power generation module 10, and the battery BA2 that performs storage due to power generation in the second power generation module 20 are provided as secondary batteries inside the rotor bearing 12, but the storage may be performed by one secondary battery. Power can be stably supplied by using power supply from these batteries together.

Further, various aspects can be adopted for a start and a stop of a detection operation of biometric information in the wearable device 100. However, for example, an aspect in which a start and a stop of the detection operation described above are determined by providing various operation buttons on an outer packaging side surface of the wearable device 100, and exclusively receiving an operation by a user may be achieved.

Further, in the description described above, the light-emitting unit 30a and the light-receiving unit 30b are separately provided in the sensor 30, but the light-emitting unit 30a and the light-receiving unit 30b may be integrally provided.

The wearable device in a specific aspect includes a power generation module including a rotor with a first direction as an axial direction of the center of rotation, and a rotor bearing including a support portion configured to rotatably support the rotor, and a sensor configured to detect biological information and provided so as to overlap the support portion in the first direction.

In the wearable device described above, in a structure in which the sensor can be supplied with power from the power generation module that generates power by rotation of the rotor, the sensor is provided so as to overlap the support portion that rotatably supports the rotor in the first direction being the axial direction of the center of rotation, and thus a position shift and the like of the sensor due to a load of rotating the rotor can be suppressed, and detection of appropriate biometric information can be maintained.

In the specific aspect, the wearable device includes a control board configured to control the sensor, wherein the rotor bearing includes an extending portion extending in the first direction and being provided in a region overlapping the support portion in the first direction, and the control board has an insertion port into which the extending portion is inserted and overlapping the sensor in the first direction. In this case, for example, a rotation operation with the extending portion as a reference can be stabilized, and a central position can be accurately positioned.

In the specific aspect, the extending portion is provided at substantially the center of the rotor bearing as viewed from the first direction. In this case, rotation of the rotor can be stabilized with the extending portion as a reference.

In the specific aspect, the wearable device includes a cushioning member provided between the control board and the sensor in cross-sectional side view. In this case, interference between the control board and the sensor can be avoided by the cushioning member.

In the specific aspect, the wearable device includes a light-transmitting member configured to cover a front surface, a first case member attached to a lower portion of the light-transmitting member, and a second case member attached to a lower portion of the first case member, wherein the power generation module is covered with the light-transmitting member and the first case member, and the sensor and the control board are covered with the first case member and the second case member. In this case, assembly can be accurately performed, and an inside state (a motion of the power generation module due to rotation of the rotor) can also be visually recognized by covering the front surface with the light-transmitting member.

In the specific aspect, the extending portion is provided at substantially the center of the first case member as viewed from the first direction. In this case, in order to stabilize rotation of the rotor with the first case member as a reference, assembly of each unit with respect to the first case member can be achieved.

In the specific aspect, the power generation module includes a plate member fixed to the first case member to form a peripheral edge portion, and the plate member is provided between the rotor and the rotor bearing. In this case, accurate assembly to other members of the rotor and the rotor bearing can be simply and reliably achieved by forming the peripheral edge portion by the plate member.

In the specific aspect, the wearable device includes a lens provided so as to overlap the sensor in the first direction, wherein the sensor is provided between the control board and the lens in the first direction, and the lens protrudes outward from the second case member in the cross-sectional side view. In this case, a sensing operation of the sensor via the lens can be appropriately performed.

In the specific aspect, the rotor bearing is a member having a first radius, and the rotor is a member having a second radius greater than the first radius. In this case, a state where the rotor is supported and is also rotated with high efficiency in the rotor bearing can be accurately maintained.

In the specific aspect, the sensor includes a light-emitting unit configured to emit irradiation light toward a living body, and a light-receiving unit configured to receive return light that is the irradiation light reflected by the living body, and at least one of the light-emitting unit or the light-receiving unit overlaps the support portion in the first direction. In this case, acquisition of intended biometric information can be reliably performed based on data about the emission of the irradiation light, and data about the reception of the return light.

What is claimed is:

1. A wearable device comprising:
a power generation module including a rotor in which an axial direction of a rotation center is a first direction, and a rotor bearing including a support portion configured to rotatably support the rotor; and
a sensor provided so as to overlap the support portion in the first direction and configured to detect biological information.

2. The wearable device according to claim 1, comprising
a control board configured to control the sensor, wherein
the rotor bearing includes an extending portion extending in the first direction and being provided in a region overlapping the support portion in the first direction, and
the control board includes an insertion port into which the extending portion is inserted, the insertion port overlapping the sensor in the first direction.

3. The wearable device according to claim 2, wherein
the extending portion is provided at substantially a center of the rotor bearing as viewed from the first direction.

4. The wearable device according to claim 2, comprising
a cushioning member provided between the control board and the sensor in cross-sectional side view.

5. The wearable device according to claim 2, comprising:
a light-transmitting member covering a front surface;
a first case member attached to a lower portion of the light-transmitting member; and
a second case member attached to a lower portion of the first case member, wherein
the power generation module is covered with the light-transmitting member and the first case member, and
the sensor and the control board are covered with the first case member and the second case member.

6. The wearable device according to claim 5, wherein
the extending portion is provided at substantially a center of the first case member as viewed from the first direction.

7. The wearable device according to claim 5, wherein
the power generation module includes a plate member fixed to the first case member to form a peripheral edge portion, and
the plate member is provided between the rotor and the rotor bearing.

8. The wearable device according to claim 5, comprising
a lens provided so as to overlap the sensor in the first direction, wherein
the sensor is provided between the control board and the lens in the first direction, and
the lens protrudes outward from the second case member in a cross-sectional side view.

9. The wearable device according to claim 1, wherein
the rotor bearing is a member having a first radius, and
the rotor is a member having a second radius greater than the first radius.

10. The wearable device according to claim 1, wherein
the sensor includes a light-emitting unit configured to emit irradiation light toward a living body, and a light-receiving unit configured to receive return light that is the irradiation light reflected by the living body, and
at least one of the light-emitting unit or the light-receiving unit overlaps the support portion in the first direction.
